(19) United States
(12) Patent Application Publication
(10) Pub. No.: US 2024/0066126 A1
(43) Pub. Date: Feb. 29, 2024

(54) COMBINATION THERAPY OF SOLID TUMORS USING CHIMERIC ANTIGEN RECEPTOR CELLS REPRESENTING ADAPTIVE AND INNATE IMMUNITY

(71) Applicant: Regen Biopharma, Inc., La Mesa, CA (US)

(72) Inventors: Thomas ICHIM, San Diego, CA (US); David KOOS, La Mesa, CA (US); Harry LANDER, La Mesa, CA (US)

(73) Assignee: Regen Biopharma, Inc., La Mesa, CA (US)

(21) Appl. No.: 18/455,544

(22) Filed: Aug. 24, 2023

Related U.S. Application Data
(60) Provisional application No. 63/400,740, filed on Aug. 24, 2022.

(51) Int. Cl. A61K 39/00; A61K 38/17; A61K 38/19

(52) U.S. Cl. CPC A61K 39/4631; A61K 38/1725; A61K 38/193; A61K 39/4614

(57) ABSTRACT

Disclosed are combination therapies for cancer utilizing the leverage of the adaptive immune system through chimeric antigen receptor (CAR) T cells, combined with leveraging the innate immune system by using CAR-macrophages (CAR-M) and CAR-natural killer (NK) cells. In some embodiments, the invention teaches the initial modification of the tumor microenvironment by administration of CAR-M and CAR-NK. The alteration of the tumor microenvironment results in reduction of barriers for CAR-T cells to enter the tumor, which allows for efficacy of CAR-T in treatment of solid tumors. In some embodiments adjuvant immunotherapies are utilized to expand immunological attack such as addition of complement, immunotherapeutic antibodies, chemotherapy and radiotherapy approaches.

### CROSS-REFERENCE TO RELATED APPLICATIONS

This application claims priority to U.S. Provisional Application No. 63/400,740, titled “Combination Therapy of Solid Tumors using Chimeric Antigen Receptor Cells Representing Adaptive and Innate Immunity”, filed Aug. 24, 2022, which is hereby incorporated by reference in its entirety.

### FIELD OF THE INVENTION

The teachings herein relate to the use of modified immunological cells for the treatment of cancer.

### BACKGROUND OF THE INVENTION

It is known that there have been a number of immunotherapeutic agents that are now used in cancer treatment, including therapeutic monoclonal antibodies (mAbs), bi-specific T-cell engagers and chimeric antigen receptors (CARs).

The field of tumor immunology was revolutionized by the clinical entry of CAR T cells. Unfortunately, successes of this approach has been primarily limited to “liquid tumors”.

A number of immunotherapeutic agents have been described for use in cancer treatment, including therapeutic monoclonal antibodies (mAbs), bi-specific T-cell engagers and chimeric antigen receptors (CARs). Chimeric antigen receptors are proteins which graft the specificity of a monoclonal antibody (mAb) to the effector function of a T-cell. Their usual form is that of a type I transmembrane domain protein with an antigen recognizing amino terminus (binder), and a transmembrane domain connected to an endodomain which transmits T-cell activation signals. The most common form of these molecules are fusions of single-chain variable fragments (scFv) derived from monoclonal antibodies, which recognize a target antigen, fused via a trans-membrane domain to a signaling endodomain. Such molecules result in activation of the T-cell in response to recognition by the scFv of its target. When T cells express such a CAR, they recognize and kill target cells that express the target antigen. CARs have been developed against various tumor-associated antigens and many are currently undergoing clinical trials. Although CAR-T cell-mediated treatment have shown success towards compact target antigens such as CD19 or GD2, chimeric antigen receptors often to fail to signal in response to antigens with bulky extracellular domains. There is therefore a need for alternative CAR T-cell approaches, capable of killing target cells expressing a large or bulky target antigen.

### SUMMARY

Preferred embodiments include methods of treating cancer comprising administration of innate and adaptive immune cells, wherein said immune cells have been modified with a chimeric antigen receptor.

Preferred methods include embodiments wherein said innate immune cells are macrophages and/or an ex vivo population of CD14 expressing cells.

Preferred methods include embodiments wherein said macrophages are M1 macrophages.

Preferred methods include embodiments wherein is macrophages are capable of producing more nitric oxide and less arginase upon activation through TLR4 as compared to control macrophages.

Preferred methods include embodiments wherein said macrophages express CD16.

Preferred methods include embodiments wherein said macrophages express CD25.

Preferred methods include embodiments wherein said macrophages express CCR7.

Preferred methods include embodiments wherein said macrophages express CD86

Preferred methods include embodiments wherein said macrophages express CD127.

Preferred methods include embodiments wherein said macrophages express interleukin-1 beta receptor.

Preferred methods include embodiments wherein said macrophages express interleukin 10 receptor.

Preferred methods include embodiments wherein said macrophages express TNF receptor p55.

Preferred methods include embodiments wherein said macrophages express TNF receptor p75.

Preferred methods include embodiments wherein said macrophages express CD215.

Preferred methods include embodiments wherein said macrophages secrete IL-1 beta.

Preferred methods include embodiments wherein said macrophages secrete IL-6.

Preferred methods include embodiments wherein said macrophages secrete IL-8.

Preferred methods include embodiments wherein said macrophages secrete IL-12.

Preferred methods include embodiments wherein said macrophages secrete IL-15.

Preferred methods include embodiments wherein said macrophages secrete IL-17.

Preferred methods include embodiments wherein said macrophages are engineered to express HMGB-1.

Preferred methods include embodiments wherein said macrophages are engineered to express IL-12.

Preferred methods include embodiments wherein said macrophages are engineered to express IL-15.

Preferred methods include embodiments wherein said macrophages are engineered to express IL-17.

Preferred methods include embodiments wherein said macrophages are engineered to express IL-18.

Preferred methods include embodiments wherein said macrophages are engineered to express IL-23.

Preferred methods include embodiments wherein said macrophages are engineered to express IL-27.

Preferred methods include embodiments wherein said macrophages are engineered to express IL-33.

Preferred methods include embodiments wherein said macrophages are engineered to express IL-37.

Preferred methods include embodiments wherein said macrophages are engineered to express a chimeric antigen receptor (CAR).

Preferred methods include embodiments wherein the CAR comprises (i) an extracellular domain comprising an antigen binding domain; (ii) a transmembrane domain; and (iii) an intracellular domain containing an intracellular signaling domain; and (b) a pharmaceutically acceptable carrier or excipient; thereby treating the cancer in the human subject.

Preferred methods include embodiments wherein the intracellular signaling domain comprises a CD3 zeta intracellular signaling domain, an Fc.epsilon.R intracellular signaling domain, an Fc.gamma.R intracellular signaling domain, or a TRIF intracellular signaling domain.

Preferred methods include embodiments wherein the intracellular domain comprises two or more intracellular signaling domains.

Preferred methods include embodiments wherein the transmembrane domain comprises a CD8a transmembrane domain or a TLR4 transmembrane domain.

Preferred methods include embodiments wherein the transmembrane domain comprises a CD8a transmembrane domain or a TLR2 transmembrane domain.

Preferred methods include embodiments wherein the transmembrane domain comprises a CD8a transmembrane domain or a TLR3 transmembrane domain.

Preferred methods include embodiments wherein the transmembrane domain comprises a CD8a transmembrane domain or a TLR5 transmembrane domain.

Preferred methods include embodiments wherein the transmembrane domain comprises a CD8a transmembrane domain or a TLR7/8 transmembrane domain.

Preferred methods include embodiments wherein the transmembrane domain comprises a CD8a transmembrane domain or a TLR9 transmembrane domain.

Preferred methods include embodiments wherein the extracellular domain further comprises a CD8a hinge domain.

Preferred methods include embodiments wherein said macrophages are CD14+ cells comprises a population of CD14+/CD16+ cells.

Preferred methods include embodiments wherein said macrophages are CD14+ cells comprises a population of CD14+/CD56+ cells.

Preferred methods include embodiments wherein said macrophages comprise a population of CD14+ monocytes, a population of CD14+ macrophages or a population of CD14+ dendritic cells.

Preferred methods include embodiments wherein said macrophages are CD14+ cells that are derived from the human subject.

Preferred methods include embodiments wherein the population of CD14+ cells is obtained from a leukapheresis sample, a blood sample, or a PBMC sample from the human subject.

Preferred methods include embodiments wherein the population of CD14+ cells is an ex vivo population of virally transduced cells.

Preferred methods include embodiments wherein the ex vivo population of CD14+ cells comprises a viral component.

Preferred methods include embodiments wherein the antigen binding domain is a single domain antibody (sdAb) or a single chain variable fragment (scFv).

Preferred methods include embodiments wherein the antigen binding domain is an anti-HER2/neu binding domain.

Preferred methods include embodiments wherein the sequence of the recombinant polynucleic acid encoding the CAR is from a viral vector.

Preferred methods include embodiments wherein the method further comprises transducing a viral vector into a population of CD14+ cells ex vivo, thereby obtaining the ex vivo population of CD14+ cells comprising the recombinant polynucleic acid with a sequence encoding a CAR.

Preferred methods include embodiments wherein the method comprises (i) extracting a blood sample from the human subject; (ii) isolating monocytes from the blood sample; and (iii) transfecting the monocytes from the blood sample with the recombinant polynucleic acid with a sequence encoding a CAR; and wherein administering comprises infusing.

Preferred methods include embodiments wherein the recombinant polynucleic acid is mRNA.

Preferred methods include embodiments wherein the ex vivo population of CD14+ cells stimulates killing of cancer cells in the human subject by T cells of the human subject.

Preferred methods include embodiments wherein the intracellular domain of the CAR is capable of inducing monocytic differentiation to M1 macrophages in the human subject.

Preferred methods include embodiments wherein the ex vivo population of CD14+ cells enhances or improves effector function of a T cell in the human subject.

Preferred methods include embodiments wherein the ex vivo population of CD14+ cells directly kills cancer cells in the human subject.

Preferred methods include embodiments wherein the ex vivo population of CD14+ cells inhibits macrophage or macrophage related cells of the human subject from promoting tumor growth.

Preferred methods include embodiments wherein the ex vivo population of CD14+ cells is phagocytic.

Preferred methods include embodiments wherein the cancer is a lymphoma.

Preferred methods include embodiments wherein the cancer is a solid tumor.

Preferred methods include embodiments wherein the cancer is a breast cancer.

Preferred methods include embodiments wherein the cancer is a metastatic cancer.

Preferred methods include embodiments wherein the cancer is an ErbB-2-expressing cancer.

Preferred methods include embodiments wherein the method further comprises administering GM-CSF, IL-2, an agent that blocks CD47 activity or an agent that induces immunogenic cell death to the human subject.

Preferred methods include embodiments wherein the method further comprises administering G-CSF.

Preferred methods include embodiments wherein the method further comprises administering M-CSF.

Preferred methods include embodiments wherein the method further comprises administering IL-2.

Preferred methods include embodiments wherein the method further comprises administering antibody to interleukin-10.

Preferred methods include embodiments wherein the method further comprises administering TNF-alpha.

Preferred methods include embodiments wherein the method further comprises administering lymphotoxin.

Preferred methods include embodiments wherein the method further comprises administering interferon alpha.

Preferred methods include embodiments wherein the method further comprises administering interferon beta.

Preferred methods include embodiments wherein the method further comprises administering interferon gamma.

Preferred methods include embodiments wherein the method further comprises administering interferon tau.

Preferred methods include embodiments wherein the method further comprises administering interferon omega.

Preferred methods include embodiments wherein the method further comprises administering IL-7.

Preferred methods include embodiments wherein the method further comprises administering IL-9.

Preferred methods include embodiments wherein the method further comprises administering IL-12.

Preferred methods include embodiments wherein the method further comprises administering IL-15.

Preferred methods include embodiments wherein the method further comprises administering IL-18.

Preferred methods include embodiments wherein the method further comprises administering IL-23.

Preferred methods include embodiments wherein the method further comprises administering IL-33.

Preferred methods include embodiments wherein the method further comprises administering C5 component of complement.

Preferred methods include embodiments wherein the method further comprises administering C3 component of complement.

Preferred methods include embodiments wherein the method further comprises administering BCG.

Preferred methods include embodiments wherein the method further comprises administering a chemotherapeutic agent.

Preferred methods include embodiments wherein the method further comprises administering an immunotherapeutic agent.

Preferred methods include embodiments wherein the method further comprises administering an immunotherapeutic agent.

Preferred methods include embodiments wherein the method further comprises administering radiotherapy.

Preferred methods include embodiments wherein said innate immune cell is a natural killer (NK) cell.

Preferred methods include embodiments wherein said NK cell is cytotoxic towards K562 cells.

Preferred methods include embodiments wherein said NK cell expresses CD25.

Preferred methods include embodiments wherein said NK cell expresses CD69.

Preferred methods include embodiments wherein said NK cell expresses CD133.

Preferred methods include embodiments wherein said NK cell expresses CD56.

Preferred methods include embodiments wherein said NK cell expresses CD16.

Preferred methods include embodiments wherein said NK cell expresses CD57.

Preferred methods include embodiments wherein said NK cell expresses Fas ligand.

Preferred methods include embodiments wherein said NK cell expresses IL-7 receptor.

Preferred methods include embodiments wherein said NK cell expresses IL-3 receptor.

Preferred methods include embodiments wherein said NK cell expresses IL-10 receptor.

Preferred methods include embodiments wherein said NK cell expresses IL-12 receptor.

Preferred methods include embodiments wherein said NK cell expresses IL-15 receptor.

Preferred methods include embodiments wherein said NK cell expresses IL-18 receptor.

Preferred methods include embodiments wherein said NK cells are derived from peripheral blood.

Preferred methods include embodiments wherein said NK cells are derived from umbilical cord blood.

Preferred methods include embodiments wherein said NK cells are derived from menstrual blood.

Preferred methods include embodiments wherein said NK cells are derived from bone marrow.

Preferred methods include embodiments wherein said NK cells are derived from mobilized peripheral blood.

Preferred methods include embodiments wherein mobilization of blood is achieved by treatment of the patient with agents that increase oxidative stress.

Preferred methods include embodiments wherein said agents that increase oxidative stress are ozone.

Preferred methods include embodiments wherein said mobilization is accomplished by treating the patient with G-CSF.

Preferred methods include embodiments wherein said mobilization is accomplished by treating the patient with GM-CSF.

Preferred methods include embodiments wherein said mobilization is accomplished by treating the patient with M-CSF.

Preferred methods include embodiments wherein said mobilization is accomplished by treating the patient with mozibil.

Preferred methods include embodiments wherein said mobilization is accomplished by treating the patient with VEGF.

Preferred methods include embodiments wherein said mobilization is accomplished by treating the patient with thrombopoietin.

Preferred methods include embodiments wherein said mobilization is accomplished by treating the patient with TNF-alpha.

Preferred methods include embodiments wherein said mobilization is accomplished by treating the patient with hepatocyte growth factor.

Preferred methods include embodiments wherein said mobilization is accomplished by treating the patient with flt-3 ligand.

Preferred methods include embodiments wherein said NK cells are generated from progenitor cells.

Preferred methods include embodiments wherein said progenitors are umbilical cord derived.

Preferred methods include embodiments wherein said progenitors are placental derived.

Preferred methods include embodiments wherein said progenitors are CD34 derived.

Preferred methods include embodiments wherein said progenitors are CD133 derived.

Preferred methods include embodiments wherein said progenitors are inducible pluripotent stem cell derived.

Preferred methods include embodiments wherein said progenitors are embryonic stem cell derived.

Preferred methods include embodiments wherein said progenitors are parthenogenic stem cell derived.

Preferred methods include embodiments wherein said progenitors are lymphoid progenitor cell derived.

Preferred methods include embodiments wherein said NK cells are engineered to express a chimeric antigen receptor (CAR).

Preferred methods include embodiments wherein the CAR comprises (i) an extracellular domain comprising an antigen binding domain; (ii) a transmembrane domain; and (iii) an intracellular domain containing an intracellular signaling domain; and (b) a pharmaceutically acceptable carrier or excipient; thereby treating the cancer in the human subject.

Preferred methods include embodiments wherein the intracellular signaling domain comprises a CD3 zeta intracellular signaling domain, an Fc.epsilon.R intracellular signaling domain, an Fc.gamma.R intracellular signaling domain, or a TRIF intracellular signaling domain.

Preferred methods include embodiments wherein the intracellular domain comprises two or more intracellular signaling domains.

Preferred methods include embodiments wherein the transmembrane domain comprises a CD8a transmembrane domain or a TLR4 transmembrane domain.

Preferred methods include embodiments wherein the transmembrane domain comprises a CD8a transmembrane domain or a TLR2 transmembrane domain.

Preferred methods include embodiments wherein the transmembrane domain comprises a CD8a transmembrane domain or a TLR3 transmembrane domain.

Preferred methods include embodiments wherein the transmembrane domain comprises a CD8a transmembrane domain or a TLR5 transmembrane domain.

Preferred methods include embodiments wherein the transmembrane domain comprises a CD8a transmembrane domain or a TLR7/8 transmembrane domain.

Preferred methods include embodiments wherein the transmembrane domain comprises a CD8a transmembrane domain or a TLR9 transmembrane domain.

Preferred methods include embodiments wherein the extracellular domain further comprises a CD8a hinge domain.

Preferred methods include embodiments wherein said NK are pre-activated by culture in IL-2.

Preferred methods include embodiments wherein said NK cells are pre-activated by culture with dendritic cells.

Preferred methods include embodiments wherein said NK cells are pre-activated by culture with monocytes.

Preferred methods include embodiments wherein said NK cells are capable of proliferating approximately one multiplication ever 15-36 hours.

Preferred methods include embodiments wherein said NK cells are capable of proliferating approximately one multiplication ever 20-30 hours.

Preferred methods include embodiments wherein said NK cells are capable of proliferating approximately one multiplication ever 15-36 hours.

Preferred methods include embodiments wherein NK cells are virally transfected.

Preferred methods include embodiments wherein the antigen binding domain is a single domain antibody (sdAb) or a single chain variable fragment (scFv).

Preferred methods include embodiments wherein the antigen binding domain is an anti-HER2/neu binding domain.

Preferred methods include embodiments wherein the sequence of the recombinant polynucleic acid encoding the CAR is from a viral vector.

Preferred methods include embodiments wherein the method further comprises transducing a viral vector into a population of CD56+ cells ex vivo, thereby obtaining the ex vivo population of CD56+ cells comprising the recombinant polynucleic acid with a sequence encoding a CAR.

Preferred methods include embodiments wherein the method comprises (i) extracting a blood sample from the human subject; (ii) isolating NK cells from the blood sample; and (iii) transfecting the NK cells from the blood sample with the recombinant polynucleic acid with a sequence encoding a CAR; and wherein administering comprises infusing.

Preferred methods include embodiments wherein the recombinant polynucleic acid is mRNA.

Preferred methods include embodiments wherein the ex vivo population of CD56+ cells stimulates killing of cancer cells in the human subject by T cells of the human subject.

Preferred methods include embodiments wherein the intracellular domain of the CAR is capable of inducing activation of NK cell in the human subject.

Preferred methods include embodiments wherein the ex vivo population of CD56+ cell enhances or improves effector function of a T cell in the human subject.

Preferred methods include embodiments wherein the ex vivo population of CD56+ cells directly kills cancer cells in the human subject.

Preferred methods include embodiments wherein adaptive immune cell is a T cell.

Preferred methods include embodiments wherein said T cell is a CD4 T cell.

Preferred methods include embodiments wherein said T cell is a CD8 T cell.

Preferred methods include embodiments wherein said T cell is an NKT cell.

Preferred methods include embodiments wherein said T cell is a double negative T cell.

Preferred methods include embodiments wherein said T cell expresses interleukin-2 receptor.

Preferred methods include embodiments wherein said T cell expresses interleukin-4 receptor.

Preferred methods include embodiments wherein said T cell expresses interleukin-7 receptor.

Preferred methods include embodiments wherein said T cell expresses interleukin-9 receptor.

Preferred methods include embodiments wherein said T cell expresses interleukin-12 receptor.

Preferred methods include embodiments wherein said T cell expresses interleukin-15 receptor.

Preferred methods include embodiments wherein said T cell expresses interleukin-17 receptor.

Preferred methods include embodiments wherein said T cell expresses interleukin-18 receptor.

Preferred methods include embodiments wherein said NK cells are engineered to express a chimeric antigen receptor (CAR).

Preferred methods include embodiments wherein the CAR comprises (i) an extracellular domain comprising an antigen binding domain; (ii) a transmembrane domain; and (iii) an intracellular domain containing an intracellular signaling domain; and (b) a pharmaceutically acceptable carrier or excipient; thereby treating the cancer in the human subject.

Preferred methods include embodiments wherein the intracellular signaling domain comprises a CD3 zeta intracellular signaling domain, an Fc.epsilon.R intracellular signaling domain, an Fc.gamma.R intracellular signaling domain, or a TRIF intracellular signaling domain.

Preferred methods include embodiments wherein the intracellular domain comprises two or more intracellular signaling domains.

Preferred methods include embodiments wherein the transmembrane domain comprises a CD8a transmembrane domain or a TLR4 transmembrane domain.

Preferred methods include embodiments wherein the transmembrane domain comprises a CD8a transmembrane domain or a TLR2 transmembrane domain.

Preferred methods include embodiments wherein the transmembrane domain comprises a CD8a transmembrane domain or a TLR3 transmembrane domain.

Preferred methods include embodiments wherein the transmembrane domain comprises a CD8a transmembrane domain or a TLR5 transmembrane domain.

Preferred methods include embodiments wherein the transmembrane domain comprises a CD8a transmembrane domain or a TLR7/8 transmembrane domain.

Preferred methods include embodiments wherein the transmembrane domain comprises a CD8a transmembrane domain or a TLR9 transmembrane domain.

Preferred methods include embodiments wherein the extracellular domain further comprises a CD8a hinge domain.

Preferred methods include embodiments wherein said T cells are pre-activated by culture in IL-2.

Preferred methods include embodiments wherein said T cells are pre-activated by culture with dendritic cells.

Preferred methods include embodiments wherein said T cells are pre-activated by culture with monocytes.

Preferred methods include embodiments wherein said T cells are capable of proliferating approximately one multiplication ever 15-36 hours.

Preferred methods include embodiments wherein said T cells are capable of proliferating approximately one multiplication ever 20-30 hours.

Preferred methods include embodiments wherein said T cells are capable of proliferating approximately one multiplication ever 15-36 hours.

Preferred methods include embodiments wherein T cells are virally transfected.

Preferred methods include embodiments wherein the antigen binding domain is a single domain antibody (sdAb) or a single chain variable fragment (scFv).

Preferred methods include embodiments wherein the antigen binding domain is an anti-HER2/neu binding domain.

Preferred methods include embodiments wherein the sequence of the recombinant polynucleic acid encoding the CAR is from a viral vector.

Preferred methods include embodiments wherein CAR-macrophages are injected initially, alone or in combination with CAR-NK.

Preferred methods include embodiments wherein CAR-T cells are administered at a timepoint after which CAR-macrophages and/or CAR-NK have modified to tumor microenvironment to allow for CAR-T cells to enter said tumor microenvironment.

Preferred methods include embodiments wherein all cells expressing CAR possess an inhibitor of NR2F6.

### DETAILED DESCRIPTION OF THE INVENTION

The invention provides means of treating cancer through inactivating the tumor microenvironment followed by administration of CAR-T cells. The invention provides the use of CAR-Macrophages and CAR-NK cells for manipulation of the tumor microenvironment in order to decrease immunesuppressinvess and thus allowing for entry of CAR-T cells into solid tumors. The role of the CAR-macrophages and CAR-NK cells are to increase propensity for Th1 immunity and allow for activity of CAR-T cells which otherwise is blunted by mediators such as PGE-2, IL-10, TGF-beta and soluble TNF-alpha receptor found in the tumor microenvironment.

The articles “a” and “an” are used herein to refer to one or to more than one (i.e., to at least one) of the grammatical object of the article. By way of example, “an element” means one element or more than one element.

As used herein, terms “comprise,” “have,” “has,” and “include” and their conjugates, as used herein, mean “including but not limited to.” While various compositions, and methods are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

“Activation”, as used herein, refers to the state of a MIL that has been sufficiently stimulated to induce detectable cellular proliferation. Activation can also be as sociated with induced cytokine production, and detectable effector functions. The term “activated MILs” refers to, among other things, MILs that are undergoing cell division.

The term “antibody,” as used herein, refers to an immunoglobulin molecule which specifically binds with an antigen. Antibodies can be intact immunoglobulins derived from natural sources or from recombinant sources and can be immunoreactive portions of intact immunoglobulins. The antibodies may exist in a variety of forms including, for example, polyclonal antibodies, monoclonal antibodies, Fv, Fab and F(ab)2, as well as single chain antibodies and humanized antibodies.

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, scFv antibodies, and multispecific antibodies formed from antibody fragments.

The term “antigen” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the embodiments include, but are not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “anti-tumor effect” as used herein, refers to a biological effect that can be manifested by a decrease in tumor volume, a decrease in the number of tumor cells, a decrease in the number of metastases, an increase in life expectancy, or amelioration of various physiological symptoms associated with the cancerous condition. An “anti-tumor effect” can also be manifested by the ability of the peptides, polynucleotides, cells and antibodies to prevent the occurrence of tumor in the first place.

The term “auto-antigen” means any self-antigen which is mistakenly recognized by the immune system as being foreign. Auto-antigens comprise, but are not limited to, cellular proteins, phosphoproteins, cellular surface proteins, cellular lipids, nucleic acids, glycoproteins, including cell surface receptors.

As used herein, the term “autologous” is meant to refer to any material derived from the same individual to which it is later to be re-introduced into the individual.

“Allogeneic” refers to a graft derived from a different animal of the same species.

“Xenogeneic” refers to a graft derived from an animal of a different species.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. Cancers that may be treated include tumors that are not vascularized, or not yet substantially vascularized, as well as vascularized tumors. The cancers may include non-solid tumors (such as hematological tumors, for example, myeloma, leukemias and lymphomas) or may include solid tumors. Types of cancers to be treated with the CARs as described herein include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

“Co-stimulatory ligand,” as the term is used herein, includes a molecule on an antigen presenting cell (e.g., an aAPC, dendritic cell, B cell, and the like) that specifically binds a cognate co-stimulatory molecule on a MIL, thereby providing a signal which, in addition to the primary signal provided by, for instance, binding of a TCR/CD3 complex with an MHC molecule loaded with peptide, mediates a MIL response, including, but not limited to, proliferation, activation, differentiation, and the like. A co-stimulatory ligand can include, but is not limited to, CD7, B7-1 (CD80), B7-2 (CD86), PD-L1, PD-L2, 4-1BBL, OX40L, inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a MIL, such as, but not limited to, CD27, CD28, 4-1BB, OX40, CD30, CD40, PD-1, ICOS, lymphocyte function-associated antigen-1 (LFA-1), CD2, CD7, LIGHT, NKG2C, B7-H3, and a ligand that specifically binds with CD83.

A “co-stimulatory molecule” refers to the cognate binding partner on a MIL that specifically binds with a co-stimulatory ligand, thereby mediating a co-stimulatory response by the MIL, such as, but not limited to, proliferation. Co-stimulatory molecules include, but are not limited to an MHC class I molecule, BTLA and a Toll ligand receptor.

A “co-stimulatory signal”, as used herein, refers to a signal, which in combination with a primary signal, such as TCR/CD3 ligation, leads to MIL proliferation and/or upregulation or downregulation of key molecules.

A “disease” is a state of health of a subject wherein the subject cannot maintain homeostasis, and wherein if the disease is not ameliorated then the animal's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

“Homologous” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are homologous at that position. The percent of homology between two sequences is a function of the number of matching or homologous positions shared by the two sequences divided by the number of positions compared .times.100. For example, if 6 of 10 of the positions in two sequences are matched or homologous then the two sequences are 60% homologous. By way of example, the DNA sequences ATTGCC and TATGGC share 50% homology. Generally, a comparison is made when two sequences are aligned to give maximum homology.

The term “immunoglobulin” or “Ig,” as used herein is defined as a class of proteins, which function as antibodies. Antibodies expressed by B cells are sometimes referred to as the BCR (B cell receptor) or antigen receptor. The five members included in this class of proteins are IgA, IgG, IgM, IgD, and IgE.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

As used herein, the following abbreviations for the commonly occurring nucleic acid bases are used. “A” refers to adenosine, “C” refers to cytosine, “G” refers to guanosine, “T” refers to thymidine, and “U” refers to uridine.

A “Lentivirus” as used herein refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses. Vectors derived from lentiviruses offer the means to achieve significant levels of gene transfer in vivo.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “overexpressed” tumor antigen or “overexpression” of the tumor antigen is intended to indicate an abnormal level of expression of the tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art.

“Parenteral” administration of an immunogenic composition includes, e.g., subcutaneous (s.c.), intravenous (i.v.), intramuscular (i.m.), or intrasternal injection, or infusion techniques.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

A classical chimeric antigen receptor (CAR) is a chimeric type I trans-membrane protein which connects an extracellular antigen-recognizing domain (binder) to an intracellular signaling domain (endodomain). The binder is typically a single-chain variable fragment (scFv) derived from a monoclonal antibody (mAb), but it can be based on other formats which comprise an antibody-like antigen binding site. A spacer domain is usually necessary to isolate the binder from the membrane and to allow it a suitable orientation. A common spacer domain used is the Fc of IgG1. More compact spacers can suffice e.g. the stalk from CD8a and even just the IgG1 hinge alone, depending on the antigen. A trans-membrane domain anchors the protein in the cell membrane and connects the spacer to the endodomain. Early CAR designs had endodomains derived from the intracellular parts of either the .gamma. chain of the Fc.epsilon.R1 or CD3.zeta. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD3.zeta. results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 41 BB which transmit survival signals. Even more potent third generation CARs have now been described which have endodomains capable of transmitting activation, proliferation and survival signals. When the CAR binds the target-antigen, this results in the transmission of an activating signal to the T-cell it is expressed on. Thus the CAR directs the specificity and cytotoxicity of the T cell towards tumor cells expressing the targeted antigen. CARs typically therefore comprise: (i) an antigen-binding domain; (ii) a spacer; (iii) a transmembrane domain; and (iii) an intracellular domain which comprises or associates with a signaling domain. A CAR may have the general structure: Antigen binding domain-spacer domain-transmembrane domain-intracellular signaling domain (endodomain). Within the context of the current invention, CARs may be utilized for application in innate cells, such as NK and macrophages, as well as in their more classical embodiment, T cells.

The invention provides means of inducing an anti-cancer response in a mammal, comprising the steps of initially “priming” the mammal by administering an agent that causes local accumulation of CAR-macrophage. Subsequently, a tumor antigen is administered in the local area where said agents causing accumulation of antigen presenting cells is administered. A time period is allowed to pass to allow for said antigen presenting cells to traffic to the lymph nodes. Subsequently a maturation signal, or a plurality of maturation signals are administered to enhance the ability of said antigen presenting cell to activate adaptive immunity. In some embodiments of the invention activators of adaptive immunity are concurrently given, as well as inhibitors of the tumor derived inhibitors are administered to derepress the immune system.

In one embodiment priming of the patient is achieved by administration of GM-CSF subcutaneously in the area in which antigen is to be injected. Various scenarios are known in the art for administration of GM-CSF prior to administration, or concurrently with administration of antigen. The practitioner of the invention is referred to the following publications for dosage regimens of GM-CSF and also of peptide antigens. Subsequent to priming, the invention calls for administration of tumor antigen. Various tumor antigens may be utilized, in one preferred embodiment, lysed tumor cells from the same patient area utilized. Means for generation of lysed tumor cells are well known in the art and described in the following references. One example method for generation of tumor lysate involves obtaining frozen autologous samples which are placed in hanks buffered saline solution (HBSS) and gentamycin 50.mu.g/ml followed by homogenization by a glass homogenizer. After repeated freezing and thawing, particle-containing samples are selected and frozen in aliquots after radiation with 25 kGy. Quality assessment for sterility and endotoxin content is performed before freezing. Cell lysates are subsequently administered into the patient in a preferred manner subcutaneously at the local areas where DC priming was initiated. After 12-72 hours, the patient is subsequently administered with an agent capable of inducing maturation of DC. Agents useful for the practice of the invention, in a preferred embodiment include BCG and HMGB1 peptide. Other useful agents include: a) histone DNA; b) imiqimod; c) beta-glucan; d) hsp65; e) hsp90; 0 HMGB-1; g) lipopolysaccharide; h) Pam3CSK4; i) Poly I: Poly C; j) Flagellin; k) MALP-2; 1) Imidazoquinoline; m) Resiquimod; n) CpG oligonucleotides; o) zymosan; p) peptidoglycan; q) lipoteichoic acid; r) lipoprotein from gram-positive bacteria; s) lipoarabinomannan from mycobacteria; t) Polyadenylic-polyuridylic acid; u) monophosphoryl lipid A; v) single stranded RNA; w) double stranded RNA; x) 852A; y) rintatolimod; z) Gardiquimod; and aa) lipopolysaccharide peptides. The procedure is performed in a preferred embodiment with the administration of IDO silencing siRNA or shRNA containing the effector sequences a) UUAUAAUGACUGGAUGUUC; b) GUCUGGUGUAUGAAGGGUU; c) CUCCUAUUUUGGUUUAUGC and d) GCAGCGUCUUUCAGUGCUU. siRNA or shRNA may be administered through various modalities including biodegradable matrices, pressure gradients or viral transfect. In another embodiment, autologous dendritic cells are generated and IDO is silenced, prior to, concurrent with or subsequent to silencing, said dendritic cells are pulsed with tumor antigen and administered systemically.

In one embodiment of the invention mature DC are modified with CAR transfection prior to administration. Culture of dendritic cells is well known in the art, for example, U.S. Pat. No. 6,936,468, issued to Robbins, et al., for the use of tolerogenic dendritic cells for enhancing tolerogenicity in a host and methods for making the same. Although the current invention aims to reduce tolerogenesis, the essential means of dendritic cell generation are disclosed in the patent. U.S. Pat. No. 6,734,014, issued to Hwu, et al., for methods and compositions for transforming dendritic cells and activating T cells. Briefly, recombinant dendritic cells are made by transforming a stem cell and differentiating the stem cell into a dendritic cell. The resulting dendritic cell is said to be an antigen presenting cell which activates T cells against MHC class I-antigen targets. Antigens for use in dendritic cell loading are taught in, e.g., U.S. Pat. No. 6,602,709, issued to Albert, et al. This patent teaches methods for use of apoptotic cells to deliver antigen to dendritic cells for induction or tolerization of T cells. The methods and compositions are said to be useful for delivering antigens to dendritic cells that are useful for inducing antigen-specific cytotoxic T lymphocytes and T helper cells. The disclosure includes assays for evaluating the activity of cytotoxic T lymphocytes. The antigens targeted to dendritic cells are apoptotic cells that may also be modified to express non-native antigens for presentation to the dendritic cells. The dendritic cells are said to be primed by the apoptotic cells (and fragments thereof) capable of processing and presenting the processed antigen and inducing cytotoxic T lymphocyte activity or may also be used in vaccine therapies. U.S. Pat. No. 6,455,299, issued to Steinman, et al., teaches methods of use for viral vectors to deliver antigen to dendritic cells. Methods and compositions are said to be useful for delivering antigens to dendritic cells, which are then useful for inducing T antigen specific cytotoxic T lymphocytes. The disclosure provides assays for evaluating the activity of cytotoxic T lymphocytes. Antigens are provided to dendritic cells using a viral vector such as influenza virus that may be modified to express non-native antigens for presentation to the dendritic cells. The dendritic cells are infected with the vector and are said to be capable of presenting the antigen and inducing cytotoxic T lymphocyte activity or may also be used as vaccines. Immune cells for use in the practice of the invention include DCs, the presence of which may be checked in the previously described method, are preferably selected from myeloid cells (such as monocytic cells and macrophages) expressing langerin, MHC (major histocompatibility complex) class II, CCR2 (chemokine (C-C motif) receptor 2), CX3CR1 and/or Grl molecules in mice; myeloid cells expressing CD14, CD16, HLA dR (human leukocyte antigen disease resistance) molecule, langerin, CCR2 and/or CX3CR1 in humans; dendritic cells expressing CD11c, MHC class II molecules, and/or CCR7 molecules; and IL-1.beta. producing dendritic cells. CD8 T cells, the presence of which may be checked in the previously described method, are preferably selected from CD3+, CD4+ and/or CD8+T lymphocytes, FOXP3 (forkhead box P3) T lymphocytes, Granzyme B/TIA (Tcell-restricted intracellular antigen) T lymphocytes, and Tc1 cells (IFN-.gamma. producing CD8+T lymphocytes). Immune cells expressing a protein that binds calreticulin, such immune cells may be selected from cells expressing at least one of the following proteins: LRP1 (Low density lipoprotein receptor-related protein 1, CD91), Ca.sup.++-binding proteins such as SCARF1 and SCARF2, MSR1 (Macrophage scavenger receptor 1), SRA, CD59 (protectin), CD207 (langerin), and THSDI (thrombospondin). There are numerous means known in the art to identify cells expressing various antigens, these include immunochemistry, immunophenotyping, flow cytometry, Elispot assays, classical tetramer staining, and intracellular cytokine staining.

Macrophages selectively phagocytose tumor cells, but this process is countered by protective molecules on tumor cells such as CD47, which binds macrophage signal-regulatory protein a to inhibit phagocytosis. Blockade of CD47 on tumor cells leads to phagocytosis by macrophages. In one embodiment of the invention CAR-MSC are administered together with an agent that blocks CD47 activity. It has been demonstrated that activation of TLR signaling pathways in macrophages synergizes with blocking CD47 on tumor cells to enhance tumor phagocytosis. Bruton's tyrosine kinase (Btk) mediates TLR signaling in macrophages. Calreticulin, previously shown to be a protein found on cancer cells that activated macrophage phagocytosis of tumors, is activated in macrophages for secretion and cell-surface exposure by TLR and Btk to target cancer cells for phagocytosis, even if the cancer cells themselves do not express calreticulin. In one embodiment of the invention TLR agonists are administered that stimulate expression of calreticulin and/or enhance macrophage phagocytosis of tumors. IL-27 induces macrophage ability to kill tumor cells in vitro and in vivo, as well as altering the tumor promoting M2/myeloid suppressor cells into tumoricidal cells. In one embodiment of the invention addition of IL-27 or compounds capable of activating the IL-27 receptor signaling are administered together with IL-27 to enhance tumor phagocytosis by macrophages.

Tumor-associated macrophages, deriving from monocytes or migrating into the tumor, are an important constituent of tumor microenvironments, which in many cases modulates tumor growth, tumor angiogenesis, immune suppression, metastasis and chemoresistance. Mechanisms of macrophage promotion of tumor growth include production of EGF, M-CSF, and VEGF. Macrophage infiltration of tumors is associated with poor prognosis in renal, melanoma, breast, pancreatic, lung, endometrial, bladder, prostate. Tumor growth are inhibited when monocytes/macrophages are ablated. There is ample evidence that many anticancer modalities currently used in the clinic have unique and distinct properties that modulate the recruitment, polarization and tumorigenic activities of macrophages in the tumor microenvironments. By manipulating tumor-associated macrophages significant impact on the clinical efficacies of and resistance to these anticancer modalities. Accordingly, in one aspect of the invention, CAR-macrophages are utilized to force the tumor microenvironment to stimulate tumor killing and inhibit macrophage or macrophage related cells from promoting tumor growth. Within the context of the invention, the use of drugs targeting tumor-associated macrophages, especially c-Fms kinase inhibitors and humanized antibodies targeting colony-stimulating factor-1 receptor, are envisioned.

Tumors mediate various effects to reprogram macrophages, these are usually mediated via IL-10 and other cytokines such as VEGF, TGF-beta, and M-CSF, which cause macrophages to lose tumor cytotoxicity and shift into tumor promoting, immune suppressive, angiogenic supporting cells. Related to tumor manipulated monocytes are myeloid derived suppressor cells, which are similar to myeloid progenitor cells, or the previously described “natural suppressor” cell.

Irradiated tissues induce a TLR-1 reprogramming of macrophages to promote tumor growth and angiogenesis. Macrophage promotion of tumor growth is seen in numerous situations, in one example, treating of tumor bearing animals with BRAF inhibitors results in upregulation of macrophage production of VEGF which accelerates tumor growth. Mechanistically, it is known that tumors produce factors such as GM-CSF which in part stimulate macrophages to produce CCL18, which promotes tumor metastasis. Additionally, the lactic acid microenvironment of the tumor has been shown to promote skewing of macrophages towards at tumor-promoting M2 type. It has been shown that lactic acid produced by tumor cells, as a by-product of aerobic or anaerobic glycolysis, possesses an essential role in inducing the expression of VEGF and the M2-like polarization of tumor-associated macrophages, specifically inducing expression of arginase 1 through a HIF-1alpha dependent pathway. Mechanistically, it is known that lactic acid in tumors is generated in a large part by lactate dehydrogenase-A (LDH-A), which converts pyruvate to lactate. siRNA silencing of LDH-A in Pan02 pancreatic cancer cells that are injected in C57BL/6 mice results in development of smaller tumors than mice injected with wild type, non-silenced Pan02 cells. Associated with the reduced tumor growth were observations of a decrease in the frequency of myeloid-derived suppressor cells (MDSCs) in the spleens of mice carrying LDH-A-silenced tumors. NK cells from LDH-A-depleted tumors had improved cytolytic function. Exogenous lactate administration was shown to increase the frequency of MDSCs generated from mouse bone marrow cells with GM-CSF and IL-6 in vitro. Furthermore, lactate pretreatment of NK cells in vitro inhibited cytolytic function of both human and mouse NK cells. This reduction of NK cytotoxic activity was accompanied by lower expression of perforin and granzyme in NK cells. The expression of NKp46 was lower in lactate-treated NK cells. Accordingly, in one embodiment of the invention, depletion of glucose levels using a ketogenic diet to lower lactate production by glycolytic tumors is utilized to augment therapeutic effects of CAR-macrophage. Utilization of ketogenic diet has been previously described for immune modulation, and cancer therapy. Specific quantification of intratumoral lactate and its manipulation has been described and incorporated by reference. Potentiation of chemotherapeutic and radiotherapeutic effects by ketogenic diets have been reported and techniques are incorporated by reference for use with the current CAR-macrophage invention. Suppression of tumor growth and activity induced by ketogenic diet may be augmented by addition of hyperbaric oxygen, thus in one embodiment of the invention, the utilization of oxidative therapies, as disclosed in references incorporated, together with ketogenic diet is utilized to augment therapeutic efficacy of CAR-macrophage.

Not only has it been well known that monocytes and macrophages infiltrate tumors and appear to support tumor growth through growth factor production and secretion of angiogenic agents, but suggestions have been made that tumors themselves, as part of the epithelial mesenchymal transition may actually differentiate into monocytes in part associated with TGF-beta production. Specifically, a study reported performing gene-profiling analysis of mouse mammary EpRas tumor cells that had been allowed to adopt an epithelial to mesenchymal transition program after long-term treatment with TGF-.beta.1 for 2 weeks. While the treated cells acquired traits of mesenchymal cell differentiation and migration, gene analysis revealed another cluster of induced genes, which was specifically enriched in monocyte-derived macrophages, mast cells, and myeloid dendritic cells, but less in other types of immune cells. Further studies revealed that this monocyte/macrophage gene cluster was enriched in human breast cancer cell lines displaying an EMT or a Basal B profile, and in human breast tumors with EMT and undifferentiated (ER-/PR-) characteristics. The plasticity of tumor cells to potentially monocytic lineages should come as no surprise given that tumor cells have been shown to differentiate directly into pericytes, and endothelial cells/vascular channels. Dopamine possesses antiangiogenic effects as well as myeloprotective effects, in one embodiment of the invention addition of dopamine to the CAR-macrophage treatment is disclosed. Vinblastine is a widely used chemotherapeutic agent that has been demonstrated to induce dendritic cell maturation. In one embodiment of the invention CAR-macrophage are utilized together with vinblastine therapy to induce augmented anticancer activity. Oxiplatin and anthracyclines have been demonstrated to not only directly kill tumor cells but also stimulate T cell immunity against tumor cells. It was demonstrated that these agents induce a rapid and prominent invasion of interleukin (IL)-17-producing .gamma..delta. (V.gamma.4(+) and V.gamma.6(+)) T lymphocytes (.gamma.6 T17 cells) that precedes the accumulation of CD8 CTLs within the tumor bed. In T cell receptor .delta.(−/−) or V.gamma.4/6(−/−) mice, the therapeutic efficacy of chemotherapy was reduced and furthermore no IL-17 was produced by tumor-infiltrating T cells, and CD8 CTLs did not invade the tumor after treatment. Although .gamma..delta. Th17 cells could produce both IL-17A and IL-22, the absence of a functional IL-17A-IL-17R pathway significantly reduced tumor-specific T cell responses elicited by tumor cell death, and the efficacy of chemotherapy in four independent transplantable tumor models. The adoptive transfer of .gamma..delta. T cells to naive mice restored the efficacy of chemotherapy in IL-17A(−/−) hosts. The anticancer effect of infused .gamma..delta. T cells was lost when they lacked either IL-1R1 or IL-17A. Intratumoral injection of dendritic cells stimulates antitumor immunity in vivo in clinical situations, suggesting that modulating the antigen presenting cell in the tumor microenvironment will induce an antitumor response. Administration of radiotherapy to tumors to induce immunogenic cell death, followed by intratumoral administration of DC has been demonstrated to result in enhanced antigen presentation, accordingly, this technique may be modified to enhance effects of CAR-macrophages. The induction of immunity to tumors in the present invention is associated with the unique nature of: a) ongoing basal cell death within the tumor; and b) cell death induced by chemotherapy, radiotherapy, hyperthermia, or otherwise induced cell death. Cell death can be classified according to the morphological appearance of the lethal process (that may be apoptotic, necrotic, autophagic or associated with mitosis), enzymological criteria (with and without the involvement of nucleases or distinct classes of proteases, like caspases), functional aspects (programmed or accidental, physiological or pathological) or immunological characteristics (immunogenic or non-immunogenic). Cell death is defined as “immunogenic” or “immune stimulatory” if dying cells that express a specific antigen (for example a tumor associated antigen, phosphotidyl serine, or calreticulin), yet are uninfected (and hence lack pathogen-associated molecular patterns), and are injected subcutaneously into mice, in the absence of any adjuvant, cause a protective immune response against said specific antigen. Such a protective immune response precludes the growth of living transformed cells expressing the specific antigen injected into mice. When cancer cells succumb to an immunogenic cell death (or immunogenic apoptosis) modality, they stimulate the immune system, which then mounts a therapeutic anti-cancer immune response and contributes to the eradication of residual tumor cells. Conversely, when cancer cells succumb to a non-immunogenic death modality, they fail to elicit such a protective immune response. Regardless of the types of cell death that are ongoing, the tumor derived immune suppressive molecules contribute to general inhibition or inability of the tumor to be eliminated. Within the practice of the invention, CAR-macrophage are administered concurrently, prior to, or subsequent to administration of an agent that induces immunogenic cell death in a patient. Methods of determining whether compounds induce immunogenic cell death are known in the art and include the following, which was described by Zitvogel et al. (a) treating the cells, the mammalian cells and inducing the cell death or apoptosis, typically of mammalian cancer cells capable of expressing calreticulin (CRT), by exposing said mammalian cells to a particular drug (the test drug), for example 18 hours; (b) inoculating (for example intradermally) the dying mammalian cells from step (a) in a particular area (for example a flank) of the mammal, typically a mouse, to induce an immune response in this area of the mammal; (c) inoculating (for example intradermally) the minimal tumorigenic dose of syngeneic live tumor cells in a distinct area (for example the opposite flank) from the same mammal, for example 7 days after step (b); and (d) comparing the size of the tumor in the inoculated mammal with a control mammal also exposed to the minimal tumorigenic dose of syngeneic live tumor cells of step (c) [for example a mouse devoid of T lymphocyte], the stabilization or regression of the tumor in the inoculated mammal being indicative of the drug immunogenicity. Other in vitro means are available for assessing the ability of various drugs or therapeutic approaches to induce immunogenic cell death. Specific characteristics to assess when screening for immunogenic cell death include: a) ability to induce dendritic cell maturation in vitro; b) ability to activate NK cells; and c) ability to induce activation of gamma delta T cells or NKT cells. Specific drugs known to induce immunogenic cell death include oxiplatine and anthracyclines, as well as radiotherapy, and hyperthermia. In the case of chemotherapies, certain chemotherapies that activate TLR4 through induction of HMGB1 have been observed to function suboptimally in patients that have a TLR4 polymorphism, thus suggesting actual contribution of TLR activation as a means of chemotherapy inhibition of cancer. Additionally, oncoviruses or oncolytic viruses are known to induce immunogenic cell death and may be useful for the practice of the invention. The CAR-macrophage disclosed in the invention may be utilized in combination with conventional immune modulators including BCG, CpG DNA, interferon alpha, tumor bacterial therapy, checkpoint inhibitors, Treg depleting agents, and low dose cyclophosphamide.

In one embodiment of the invention CAR-macrophage cells are generated with specificity towards ROBO-4. Numerous means of generating CAR-T cells are known in the art, which are applied to CAR-macrophage. In one embodiment of the invention FMC63-28z CAR (Genebank identifier HM852952.1), is used as the template for the CAR except the anti-CD19, single-chain variable fragment sequence is replaced with an ROBO-4 fragment. The construct is synthesized and inserted into a pLNCX retroviral vector. Retroviruses encoding the ROBO-4-specific CAR are generated using the retrovirus packaging kit, Ampho (Takara), following the manufacturer's protocol. For generation of CAR-MACROPHAGE cells donor blood is obtained and after centrifugation on Ficoll-Hypaque density gradients (Sigma-Aldrich), PBMCs are plated at 2.times.10(6) cells/mL in cell culture for 2 hours and the adherent cells are collected. The cells were then stimulated for 2 days on a tissue-culture-treated 24-well plate containing M-CSF at a concentration of 100 ng/ml For retrovirus transduction, a 24-well plate are coated with RetroNectin (Takara) at 4.degree. C. overnight, according to the manufacturer's protocol, and then blocked with 2% BSA at room temperature for 30 min. The plate was then loaded with retrovirus supernatants at 300.mu.L/well and incubated at 37.degree. C. for 6 h. Next, 1.times.10(6) stimulated adherent cells in 1 mL of medium are added to 1 mL of retrovirus supernatants before being transferred to the pre-coated wells and cultured at 37.degree. C. for 2 d. The cells are then transferred to a tissue-culture-treated plate at 1.times.10 (6) cells/mL and cultured in the presence of 100 U/mL of recombinant human M-CSF, applying the T cell protocol but not utilizing IL-2 or antiCD3/antiCD28.

Other means of generating CARs are known in the art and incorporated by reference. For example, Groner's group genetically modified T lymphocytes and endowed them with the ability to specifically recognize cancer cells. Tumor cells overexpressing the ErbB-2 receptor served as a model. The target cell recognition specificity was conferred to T lymphocytes by transduction of a chimeric gene encoding the zeta-chain of the TCR and a single chain antibody (scFv(FRP5)) directed against the human ErbB-2 receptor. The chimeric scFv(FRP5)-zeta gene was introduced into primary mouse T lymphocytes via retroviral gene transfer. Naive T lymphocytes were activated and infected by cocultivation with a retrovirus-producing packaging cell line. The scFv(FRP5)-zeta fusion gene was expressed in >75% of the T cells. These T cells lysed ErbB-2-expressing target cells in vitro with high specificity. In a syngeneic mouse model, mice were treated with autologous, transduced T cells. The adoptively transferred scFv(FRP5)-zeta-expressing T cells caused total regression of ErbB-2-expressing tumors. The presence of the transduced T lymphocytes in the tumor tissue was monitored. No humoral response directed against the transduced T cells was observed. Abs directed against the ErbB-2 receptor were detected upon tumor lysis. Hornbach et al. constructed an anti-CEA chimeric receptor whose extracellular moiety is composed of a humanized scFv derived from the anti-CEA mAb BW431/26 and the CH2/CH3 constant domains of human IgG. The intracellular moiety consists of the gamma-signaling chain of the human Fc epsilon RI receptor constituting a completely humanized chimeric receptor. After transfection, the humBW431/26 scFv-CH2CH3-gamma receptor is expressed as a homodimer on the surface of MD45 T cells. Co-incubation with CEA+ tumor cells specifically activates grafted MD45 T cells indicated by IL-2 secretion and cytolytic activity against CEA+ tumor cells. Notably, the efficacy of receptor-mediated activation is not affected by soluble CEA up to 25 micrograms/ml demonstrating the usefulness of this chimeric receptor for specific cellular activation by membrane-bound CEA even in the presence of high concentrations of CEA, as found in patients during progression of the disease (200). These methods are described to guide one of skill in the art to practicing the invention, which in one embodiment is the utilization of CAR T cell approaches towards targeting tumor endothelium as comparted to simply targeting the tumor itself.

Targeting of mucins associated with cancers has been performed with CAR T cells by grafting the antibody that binds to the mucin with CD3 zeta chain. For the purpose of the invention, this procedure is modified for CAR-macrophage. In an older publication chimeric immune receptor consisting of an extracellular antigen-binding domain derived from the CC49 humanized single-chain antibody, linked to the CD3zeta signaling domain of the T cell receptor, was generated (CC49-zeta). This receptor binds to TAG-72, a mucin antigen expressed by most human adenocarcinomas. CC49-zeta was expressed in CD4+ and CD8+ T cells and induced cytokine production on stimulation. Human T cells expressing CC49-zeta recognized and killed tumor cell lines and primary tumor cells expressing TAG-72. CC49-zeta T cells did not mediate bystander killing of TAG-72-negative cells. In addition, CC49-zeta T cells not only killed FasL-positive tumor cells in vitro and in vivo, but also survived in their presence, and were immunoprotective in intraperitoneal and subcutaneous murine tumor xenograft models with TAG-72-positive human tumor cells. Finally, receptor-positive T cells were still effective in killing TAG-72-positive targets in the presence of physiological levels of soluble TAG-72, and did not induce killing of TAG-72-negative cells under the same conditions.

For clinical practice of the invention several reports exist in the art that would guide the skilled artisan as to concentrations, cell numbers, and dosing protocols useful. While in the art CAR T cells have been utilized targeting surface tumor antigens, the main issue with this approach is the difficulty of T cells to enter tumors due to features specific to the tumor microenvironment. These include higher interstitial pressure inside the tumor compared to the surroundings, acidosis inside the tumor, and expression in the tumor of FasL which kills activated T cells. Accordingly the invention seeks to more effectively utilize CAR-macrophage cells by directly targeting them to tumor endothelium, which is in direct contact with blood and therefore not susceptible to intratumoral factors the limit efficacy of conventional T cell therapies. In other embodiments CAR-macrophage are targeting to tumor antigens.

In one embodiment of the invention, protocols similar to Kershaw et al. are utilized with the exception that tumor endothelial antigens are targeted as opposed to conventional tumor antigens. Such tumor endothelial antigens include CD93, TEM-1, VEGFR1, and survivin. Antibodies can be made for these proteins, methodologies for which are described in U.S. Pat. Nos. 5,225,539, 5,585,089, 5,693,761, and 5,639,641. In one example that may be utilized as a template for clinical development, T cells with reactivity against the ovarian cancer-associated antigen alpha-folate receptor (FR) were generated by genetic modification of autologous T cells with a chimeric gene incorporating an anti-FR single-chain antibody linked to the signaling domain of the Fc receptor gamma chain. Patients were assigned to one of two cohorts in the study. Eight patients in cohort 1 received a dose escalation of T cells in combination with high-dose interleukin-2, and six patients in cohort 2 received dual-specific T cells (reactive with both FR and allogeneic cells) followed by immunization with allogeneic peripheral blood mononuclear cells. Five patients in cohort 1 experienced some grade 3 to 4 treatment-related toxicity that was probably due to interleukin-2 administration, which could be managed using standard measures. Patients in cohort 2 experienced relatively mild side effects with grade 1 to 2 symptoms. No reduction in tumor burden was seen in any patient. Tracking 111In-labeled adoptively transferred T cells in cohort 1 revealed a lack of specific localization of T cells to tumor except in one patient where some signal was detected in a peritoneal deposit. PCR analysis showed that gene-modified T cells were present in the circulation in large numbers for the first 2 days after transfer, but these quickly declined to be barely detectable 1 month later in most patients. Similar CAR-T clinical studies have been reported for neuroblastoma, B cell malignancies, melanoma, ovarian cancer, renal cancer, mesothelioma, and head and neck cancer.

In one embodiment of the invention, PBMCs are derived from leukapheresis and CD14 monocytes are collected by MACS. After 3 days of culture, M-CSF at 100 ng/ml plasmid encoding the chimeric CAR-macrophage recognizing tumor specific antigen and subsequently selected for gene integration by culture in G418. In another embodiment the generation of dual-specific T cells is performed, stimulation of allogeneic monocytic cells is achieved by coculture of patient PBMCs with irradiated (5,000 cGy) allogeneic donor PBMCs from cryopreserved apheresis product (mixed lymphocyte reaction). The MHC haplotype of allogeneic donors is determined before use, and donors that differed in at least four MHC class I alleles from the patient are used. Culture medium consisted of AimV medium (Invitrogen, Carlsbad, Calif.) supplemented with 5% human AB.sup.-serum (Valley Biomedical, Winchester, Va.), penicillin (50 units/mL), streptomycin (50 mg/mL; Bio Whittaker, Walkersville, Md.), amphotericin B (Fungizone, 1.25 mg/mL; Biofluids, Rockville, Md.), L-glutamine (2 mmol/L; Mediatech, Herndon, Va.), and human recombinant IL-2 (Proleukin, 300 IU/mL; Chiron). Mixed lymphocyte reaction consisted of 2.times.10.sup.6 patient monocytes and 1.times.10.sup.7 allogeneic stimulator PBMCs in 2 mL AimV per well in 24-well plates. Between 24 and 48 wells are cultured per patient for 3 days, at which time transduction is done by aspirating 1.5 mL of medium and replacing with 2.0 mL retroviral supernatant containing 300 IU/mL IL-2, 10 mmol/L HEPES, and 8.mu.g/mL polybrene (Sigma, St. Louis, Mo.) followed by covering with plastic wrap and centrifugation at 1,000.times.g for 1 hour at room temperature. After overnight culture at 37.degree. C./5% CO.sub.2, transduction is repeated on the following day, and then medium was replaced after another 24 hours. Cells are then resuspended at 1.times.10.sup.6/mL in fresh medium containing 0.5 mg/mL G418 (Invitrogen) in 175-cm.sup.2 flasks for 5 days before resuspension in media lacking G418. ‘Cells are expanded to 2.times.10.sup.9 and then restimulated with allogeneic PBMCs from the same donor to enrich for T cells specific for the donor allogeneic haplotype. Restimulation is done by incubating patient T cells (1.times.10.sup.6/mL) and stimulator PBMCs (2.times.10.sup.6/mL) in 3-liter Fenwall culture bags in AimV+additives and IL-2 (no G418). Cell numbers were adjusted to 1.times.10.sup.6/mL, and IL-2 was added every 2 days, until sufficient numbers for treatment were achieved.

The present invention relates to a strategy of adoptive cell transfer of monocytes or DC transduced to express a chimeric antigen receptor (CAR). CARs are molecules that combine antibody-based specificity for a desired antigen (e.g., tumor endothelial antigen) with a T cell receptor-activating intracellular domain to generate a chimeric protein that exhibits a specific anti-tumor endothelium cellular immune activity. In one embodiment the present invention relates generally to the use of monocytes or DC cells genetically modified to stably express a desired CAR that possesses high affinity towards tumor associated endothelium. Monocytes or DC cells expressing a CAR are referred to herein as CAR-MACROPHAGE cells or CAR modified DC cells. Preferably, the cell can be genetically modified to stably express an antibody binding domain on its surface, conferring novel antigen specificity that is MHC independent. In some instances, the monocyte or DC cell is genetically modified to stably express a CAR that combines an antigen recognition domain of a specific antibody with an intracellular domain of the CD3-zeta chain or Fc.gamma.RI protein into a single chimeric protein. In another embodiment, TLR signaling molecules are engineered in the intracellular portion of the CAR, said molecules include TRIF, TRADD, and MyD99. In one embodiment, the CAR of the invention comprises an extracellular domain having an antigen recognition domain, a transmembrane domain, and a cytoplasmic domain. In one embodiment, the transmembrane domain that naturally is associated with one of the domains in the CAR is used. In another embodiment, the transmembrane domain can be selected or modified by amino add substitution to avoid binding of such domains to the transmembrane domains of the same or different surface membrane proteins to minimize interactions with other members of the receptor complex. Preferably, the transmembrane domain is the CD8a hinge domain. With respect to the cytoplasmic domain, the CAR of the invention can be designed to comprise the CD80 and/or CD86 and/or CD40L and/or OX40L signaling domain by itself or be combined with any other desired cytoplasmic domain(s) useful in the context of the CAR of the invention. In one embodiment, the cytoplasmic domain of the CAR can be designed to further comprise the signaling domain of MyD88. For example, the cytoplasmic domain of the CAR can include but is not limited to CD80 and/or CD86 and/or CD40L and/or OX40L signaling modules and combinations thereof. In another embodiment of the invention inhibition of TGF-beta is performed either by transfection with an shRNA possessing selectively towards TGF-beta or by constructing the CAR to possess a dominant negative mutant of TGF-beta receptor. This would render the CAR-macrophage cell resistant to inhibitory activities of the tumors. Accordingly, the invention provides CAR-macrophage cells and methods of their use for adoptive therapy. In one embodiment, the CAR-macrophage cells of the invention can be generated by introducing a lentiviral vector comprising a desired CAR, for example a CAR comprising anti-CD19, CD8a hinge and transmembrane domain, and MyD88, into the cells. The CAR-macrophage cells of the invention are able to replicate in vivo resulting in long-term persistence that can lead to sustained tumor control.

One skilled in the art will appreciate that these methods, compositions, and cells are and may be adapted to carry out the objects and obtain the ends and advantages mentioned, as well as those inherent therein. The methods, procedures, and devices described herein are presently representative of preferred embodiments and are exemplary and are not intended as limitations on the scope of the invention. Changes therein and other uses will occur to those skilled in the art which are encompassed within the spirit of the invention and are defined by the scope of the disclosure. It will be apparent to one skilled in the art that varying substitutions and modifications may be made to the invention disclosed herein without departing from the scope and spirit of the invention. Those skilled in the art recognize that the aspects and embodiments of the invention set forth herein may be practiced separate from each other or in conjunction with each other. Therefore, combinations of separate embodiments are within the scope of the invention as disclosed herein. All patents and publications mentioned in the specification are indicative of the levels of those skilled in the art to which the invention pertains. All patents and publications are herein incorporated by reference to the same extent as if each individual publication was specifically and individually indicated to be incorporated by reference. According to one embodiment of the present invention, the chimeric antigen receptor may include two intracellular signaling domains. For example, the chimeric antigen receptor may include a first intracellular signaling domain linked to the transmembrane domain and a second intracellular signaling domain linked to a terminal of the first intracellular signaling domain that is not linked with the transmembrane domain. According to a more specific embodiment, the first intracellular signaling domain may include the whole or a portion of any one selected from the group consisting of OX40 (CD134), OX40 ligand (OX40L, CD252), 4-1BB (CD137), CD28, DAP10, CD3-zeta (CD3) and DAP12, and the second intracellular signaling domain may include the whole or a portion of any one selected from the group consisting of OX40 ligand, CD3-zeta and DAP12. In this case, at least one of the first intracellular signaling domain and the second intracellular signaling domain includes the whole or a portion of OX40 ligand. For example, the chimeric antigen receptor may include a first intracellular signaling domain containing the whole or a portion of OX40 ligand and a second intracellular signaling domain containing the whole or a portion of any one selected from CD3-zeta and DAP12. Further, for example, the chimeric antigen receptor may include a first intracellular signaling domain containing the whole or a portion of any one selected from the group consisting of CD3-zeta and DAP12 and a second intracellular signaling domain containing the whole or a portion of OX40 ligand. According to another embodiment of the present invention, the chimeric antigen receptor may include three intracellular signaling domains. For example, the chimeric antigen receptor may include: a tirst intracellular signaling domain linked to the transmembrane domain; a second intracellular signaling domain linked to a terminal of the first intracellular signaling domain that is not linked with the transmembrane domain; and a third intracellular signaling domain linked to a terminal of the second intracellular signaling domain that is not linked with the first intracellular signaling domain. According to a more specific embodiment, the first intracellular signaling domain may include the whole or a portion of any one selected from the group consisting of 4-1BB, OX40, OX40 ligand, CD28 and DAP10, the second intracellular signaling domain may include the whole or a portion of any one selected from the group consisting of OX40 ligand, OX40 and 4-1BB, and the third intracellular signaling domain may include the whole or a portion of any one selected from the group consisting of OX40 ligand, CD3-zeta and DAP12. In such a case, at least one of the first intracellular signaling domain, the second intracellular signaling domain and the third intracellular signaling domain may include the whole or a portion of OX40 ligand. In another aspect, the present invention may provide a chimeric antigen receptor, which includes: a first intracellular signaling domain containing the whole or a portion of any one selected from the group consisting of CD28 and 4-1BB; a second intracellular signaling domain containing the whole or a portion of any one selected from the group consisting of OX40 ligand, OX40 and 4-1BB; and a third intracellular signaling domain containing the whole or a portion of CD3-zeta, wherein the first, second and third intracellular signaling domains are arranged in order from the cell membrane toward the inside of the cell. According to one embodiment of the present invention, the above respective domains may be directly linked to one another or may be linked by a linker. According to one embodiment of the present invention, the chimeric antigen receptor may further include: a transmembrane domain linked to the first intracellular signaling domain; a spacer domain linked to the transmembrane domain; and an extracellular domain linked to the spacer domain. In addition, the chimeric antigen receptor may further include a signal sequence linked to the extracellular domain. According to one embodiment of the present invention, the above respective domains may be directly linked to one another or may be linked by a linker.

According to one embodiment of the present invention, the extracellular domain is a domain for specifically binding with an antibody or specifically recognizing an antigen, for example, an Fc receptor, an antigen-binding fragment of an antibody such as a single-chain variable fragment (ScFv), NK receptor (natural cytotoxicity receptor), NKG2D, 2B4 or DNAM-1, etc. Thus, in the present disclosure, the term “extracellular domain” is used with the same meanings as the “antigenic recognition site”, “antigen-binding fragment” and/or “antibody binding site.” The chimeric antigen receptor according to an embodiment of the present invention may include an Fc receptor as the extracellular domain, and therefore, can be used along with a variety of antibodies depending on cell types of cancer to be treated. According to one embodiment, the Fc receptor may include any one selected from the group consisting of CD16, CD32, CD64, CD23 and CD89, and variants thereof. According to a more specific embodiment, the Fc receptor may include CD16 or variants thereof, and most specifically, may include the whole or a portion of CD16 V158 variant (CD16V). According to another embodiment, the chimeric antigen receptor of the present invention may include, as the extracellular domain, an antigen-binding fragment of an antibody which directly recognizes the antigen without co-administration along with the antibody. According to one embodiment, the antigen-binding fragment may be an Fab fragment, F(ab′) fragment, F(ab′)2 fragment or Fv fragment. According to one embodiment of the present invention, the antibody may be any one of various types of antibodies capable of binding antigen-specifically. For example, the antibody may be one in which one light chain and one heavy chain are bonded with each other, or one in which two light chains and two heavy chains are bonded with each other. For example, when two light chains and two heavy chains are bonded with each other, the antibody may be one in which the first unit including the first light chain and the first heavy chain bonded with each other and the second unit including the second light chain and the second heavy chain bonded with each other are combined with each other. The bond may be a disulfide bond, but it is not limited thereto. According to an embodiment of the present invention, the above two units may be the same as or different from each other. For example, the first unit including the first light chain and the first heavy chain and the second unit including the second light chain and the second heavy chain may be the same as or different from each other. As such, an antibody prepared to recognize two different antigens by the first unit and the second unit, respectively, is commonly referred to as a ‘bispecific antibody’ in the related art. In addition, for example, the antibody may be one in which the above three or more units are combined with one another. The antigen-binding fragment of the present invention may be derived from various types of antibodies as described above, but it is not limited thereto.

According to another embodiment of the present invention, the extracellular domain used herein may be a NK receptor (natural cytotoxicity receptor). According to a specific embodiment, the NK receptor may include NKp46, NKp30, NKp44, NKp80 and NKp65 receptors, but it is not limited thereto.

According to one embodiment, the signal sequence may include the whole or a portion of CD16. According to another embodiment, the extracellular domain may include the whole or a portion of CD16 V158 variant (CD16V). According to another embodiment, the spacer domain may include the whole or a portion of any one selected from the group consisting of CD8.alpha. (CD8-alpha) and CD28. According to another embodiment, the transmembrane domain may include the whole or a portion of any one selected from the group consisting of CD8.alpha. and CD28.

According to another aspect, the present invention provides immune cells (e.g., NK cells) to express the above-described chimeric antigen receptor according to the present invention.

The immune cells of the present invention may exhibit toxicity to tumor cells. It is determined that the chimeric antigen receptor according to the invention exhibits specific toxicity to what types of tumor cells depending on what types of antibodies are combined with the extracellular domains. Therefore, the types of tumor cells, to which the immune cells expressing the chimeric antigen receptor according to the present invention may exhibit specific toxicity, are not particularly limited. According to one embodiment, when the immune cells (e.g., NK cells) of the present invention are used along with rituximab, the cells may exhibit toxicity to malignant lymphoma cells. For example, the malignant lymphoma cells may express CD20. Further, for example, the malignant lymphoma may be B-cell lymphoma.

According to another aspect, the invention further provides a pharmaceutical composition for prevention or treatment of tumor or tumor metastasis, which includes the immune cells (e.g., NK cells) expressing the above-described chimeric antigen receptor according to the present invention, in the number of 2 to 7.5 times the number of tumor cells (e.g., malignant lymphoma cells) in a subject to be treated (‘treatment target’). According to one embodiment of the present invention, the number of immune cells (e.g., NK cells) included in the pharmaceutical composition of the present invention in a single dose may range from 0.75 to 10 times the number of tumor cells (e.g., malignant lymphoma cells) in the treatment target. For example, the number of the immune cells (e.g., NK cells) in a single dose may range from 2 to 7.5 times the number of tumor cells (e.g., malignant lymphoma cells) in the treatment target. In some embodiments of the invention NK cell lines are engineered to express CAR. In one embodiment the NK cell line is NK-92.

Sequential administration of CAR-NK and CAR-macrophages are disclosed. In some embodiments addition of CAR-T cells is performed last.

1. A method of treating cancer comprising administration of innate and adaptive immune cells, wherein said immune cells have been modified with a chimeric antigen receptor (CAR).

2. The method of claim 1, wherein said innate immune cells are macrophages and/or an ex vivo population of CD14 expressing cells.

3. The method of claim 2, wherein said macrophages are engineered to express a chimeric antigen receptor (CAR).

4. The method of claim 3, wherein the CAR comprises (i) an extracellular domain comprising an antigen binding domain; (ii) a transmembrane domain; and (iii) an intracellular domain containing an intracellular signaling domain; and (b) a pharmaceutically acceptable carrier or excipient; thereby treating the cancer in the human subject.

5. The method of claim 3, wherein the intracellular signaling domain comprises a CD3 zeta intracellular signaling domain, an Fc.epsilon.R intracellular signaling domain, an Fc.gamma.R intracellular signaling domain, or a TRIF intracellular signaling domain.

6. The method of claim 3, wherein the intracellular domain comprises two or more intracellular signaling domains.

7. The method of claim 3, wherein the transmembrane domain comprises a CD8a transmembrane domain or a TLR4 transmembrane domain.

8. The method of claim 2, wherein said macrophages are CD14+ cells comprises a population of CD14+/CD16+ cells.

9. The method of claim 2, wherein said macrophages are CD14+ cells comprises a population of CD14+/CD56+ cells.

10. The method of claim 2, wherein said macrophages comprise a population of CD14+ monocytes, a population of CD14+ macrophages or a population of CD14+ dendritic cells.

11. The method of claim 3, wherein the method comprises (i) extracting a blood sample from the human subject; (ii) isolating monocytes from the blood sample; and (iii) transfecting the monocytes from the blood sample with the recombinant polynucleic acid with a sequence encoding a CAR; and wherein administering comprises infusing.

12. The method of claim 3, wherein the recombinant polynucleic acid is mRNA.

13. The method of claim 3, wherein the ex vivo population of CD14+ cells stimulates killing of cancer cells in the human subject by T cells of the human subject.

14. The method of claim 3, wherein the intracellular domain of the CAR is capable of inducing monocytic differentiation to M1 macrophages in the human subject.

15. The method of claim 3, wherein the ex vivo population of CD14+ cells enhances or improves effector function of a T cell in the human subject.

16. The method of claim 3, wherein the ex vivo population of CD14+ cells directly kills cancer cells in the human subject.

17. The method of claim 3, wherein the ex vivo population of CD14+ cells inhibits macrophage or macrophage related cells of the human subject from promoting tumor growth.

18. The method of claim 3, wherein the ex vivo population of CD14+ cells is phagocytic.

19. The method of claim 3, wherein the method further comprises administering M-CSF.

20. The method of claim 3, wherein the method further comprises administering C5 component of complement.
